(19) United States
(12) Patent Application Publication
(10) Pub. No.: US 2019/0038860 A1
(43) Pub. Date: Feb. 7, 2019

(54) ORAL AIRWAY SECURING SYSTEMS AND METHODS

(71) Applicant: Brandon E. Lykins, Albuquerque, NM (US)

(72) Inventor: Brandon E. Lykins, Albuquerque, NM (US)

(21) Appl. No.: 15/666,892

(22) Filed: Aug. 2, 2017

(51) Int. Cl. A61M 16/04; A61M 25/10; A61M 25/00; A61M 16/20; A61M 16/08

(52) U.S. Cl. CPC A61M 16/044; A61M 25/10182; A61M 25/0097; A61M 16/0488; A61M 16/0816; A61M 16/0463; A61M 16/208

(57) ABSTRACT

Oral airway securing devices can have multifunction adapters located at the airway tube distal end. Airway tubes provide clear airways that passes through patients' mouths and, usually, to the trachea where a cuff seals the airway tube inside the patient's airway. The multifunction adapter is fixed to the end of the airway tube outside of the patient and provides an attachment point for attaching ventilation devices that can help a patient breath through the airway tube. The multifunction adapter also provides attachment points for the pilot cuff and air syringe to thereby reliably secure them. An angled hole in the multifunction adapter can hold a syringe adapter such that the pilot cuff is held in place. An air syringe attached to the syringe adapter can be held in position at an angle avoiding interference with ventilation devices such as air bags and ventilators.

### FIELD OF THE EMBODIMENTS

Embodiments are generally related to oral tracheal intubation devices, to methods of orally intubating a patient, endotracheal intubation, and to supraglottic airway devices.

### BACKGROUND

Endotracheal intubation is the placement of a tube into the trachea to thereby maintain an open airway through which a patient can breathe. Oral tracheal intubation is a type of endotracheal intubation wherein the tube is passed through the patient's mouth and into the trachea. A number of different oral tracheal intubation devices are available with oral tracheal intubation and related devices having a wide variety of names such as endotracheal tubes and supraglottic airway devices. Specific devices include the Laryngeal Mask Airway (LMA) and King LT airway. Existing oral tracheal intubation devices generally have a number of similar components including an airway tube, distal attachment, and a cuff. The cuff is inflatable and, when inflated, seals the airway tube within the patient's trachea such that fluids from the esophagus cannot enter the trachea. The distal attachment is located at the end of the airway tube farthest from the trachea and provides an interface, typically the standardized 15 mm cylinder, for attachment of ventilation devices such as ventilation bags. The cuff is typically inflated by closing an air syringe that forces air into a filling tube and then into the cuff.

In emergency situations, chaos reigns and can lead to difficulty in maintaining control over all aspects of deployed oral intubation devices and in securing the patients airway. Systems and methods that help secure the patient's airway and help maintain that airway are needed.

### SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

An oral airway securing device can have a multifunction adapter that has a distal attachment, proximal attachment, and bracket. The distal end is the section to which ventilation devices such as ventilation bags or medical ventilator can be attached Most airway securing devices, including the prior art ones and regardless of manufacturer, have a cylinder with a nominal 15 mm outer diameter as the distal end for compatibility with other equipment. The airway tube, which is the tube that is inserted through the mount and typically into the trachea, is attached to the proximal end. The bracket is a flat piece between the distal attachment and the proximal attachment. In some embodiments, the bracket has an angled hole that holds an adapter for an air syringe. In other embodiments, the syringe adapter is part of the bracket. Still, other embodiments use different means to attach the syringe adapter to the bracket.

The syringe adapter is part of a pilot cuff, which is an assembly having the syringe adapter, a one way air valve, a balloon, and an inflating tube port. In practice, an air syringe is pushed onto the syringe adapter and the plunger pushed to force air through the syringe adapter and into the balloon. The balloon fills, giving a positive indication that the inflation system is working. The air then exits the pilot cuff through an inflation tube. The inflation tube enters the airway tube through a sidewall, and exits towards the end of the airway tube where a cuff is located. As such, operating the syringe plunger causes the cuff to inflate and deflate. The cuff seals the airway tube within the patient's airway. The one way air valve keeps the cuff from deflating when the syringe is removed. Pressing the syringe onto the syringe adapter also presses the one way air valve open such that the syringe can be used to deflate the cuff. Many embodiments can use spring loaded one way air valves wherein a spring presses the valve closed.

As discussed above, the airway tube is the tube through which the patient breaths. The adapter end of the airway tube is attached to the proximal attachment of the multifunction adapter. The patient end of the airway tube is the end inserted into the patient's airway. The cuff is near the patient end and, in many embodiments, encompasses the airway tube. For example, a short inflatable cylinder having the same main axis as the patient end of the airway tube would encompass the airway tube. When inflating, the sidewalls of the cylinder move away from the sidewalls of the airway tube. Other embodiments can have cuffs that do not encompass the airway tube.

Some embodiments have an angled syringe adapter such that the air syringe is angled away from the proximal attachment. The angle helps maintain the air syringe in position while also being clear of ventilation devices attached to the proximal attachment. Generally, the angle should be at least 5 degrees, with further clearance provided by angles of 10, 15, 20, or more degrees. Certain embodiments hold the air syringe at, or near, one of 5 degree steps previously mentioned, other embodiments can hold the air syringe at an angle between two of those steps. An aspect of the air syringes angle is that the distance between the airway tube and the inflating tube port is often smaller than the distance between the airway tube and the syringe adapter. In some embodiments, an angled hole in the bracket holds a syringe adapter, in others the syringe adapter is molded into the bracket at an angle.

Some embodiments provide a rotating bracket. The center of rotation is typically the central axis of the distal and proximal attachments. In such embodiments, the distal and proximal attachments can be a first unitary body while the bracket is a second unitary body. The second unitary body can spin in a groove in the sidewall of the first unitary body. In some embodiments, the bracket can spin unless impeded by some other element of the airway securing device, such as the inflating tube which would wrap around the airway tube. The angle of rotation would therefore be limited but, depending on the design, can exceed 80 degrees.

As discussed above, the inflating, tube passes through the sidewall of the airway tube. Ideally, the sidewall holes are snug to the inflating tube such that air and fluids cannot easily pass through the sidewalls alongside the inflating tube. Certain embodiments minimize the amount of inflation tube that is outside the airway tube. For example, the inflation tube can pass through the sidewall within 50 mm of the bracket. Many airway securing devices also have a suction tube passing through the sidewall and down to the patient end of the airway tube. The end of the suction tube is sometimes called a suction port and provides a means for drawing fluids from a patients airway.

When not flexed, the airway tube typically has a curve. The curve, hopefully, approximates the curve the tube would have when intubating a patient. As such, the airway tube has a convex side and a concave side. The inflating tube can pass though the sidewall along the convex side or along the concave side. Such placement can minimize the length of inflating tube between sidewall entry point and the pilot cuff balloon depending on whether the airway tube curves toward or away from the syringe adapter. Many embodiments also have depth markers on the airway tube to indicate the distance from the patient end to the depth marker.

A removable stylet is often installed with the airway tube to help with the placement of the airway tube in a patients airway. The stylet is posable meaning that it can be bent into a shape and will hold that shape. With the stylet inside the airway tube, a care giver can bend the airway tube into a shape that hopefully adds precision and reduces error to the task of placing the airway tube into a patient's airway. The embodiments can use a stylet having a device, such as a syringe holder, that holds the air syringe. The stylet can hold the air syringe in engagement with the syringe adapter. Pulling the stylet from the airway tube can also cause the air syringe to disengage from the syringe adapter because the stylet pulls the syringe out of engagement. The syringe is removable held when it can be removed from the stylet.

A syringe tab can hold the plunger open such that the syringe tab must be removed before the air syringe can force air into the pilot cuff. A thinned section of the syringe tab can be easily broken to facilitate removal of the syringe tab and to positively indicate that the air syringe has been used. Alternatively, the syringe tab can be designed to break when the plunger is pressed to positively indicate a used air syringe without also impeding the use of the air syringe.

It is a further aspect of the embodiments to pass the patient end of an airway tube into a patient's mouth and thence to the patient's trachea. The airway tube has a patient end and an adapter end. The adapter end is attached to the proximal attachment of a multifunction adapter that also has a distal attachment and a bracket with the bracket between the distal attachment and the proximal attachment. Air passing into the distal attachment passes though the bracket, through the proximal attachment, and into the airway tube. Forcing air into a pilot cuff, inflating tube, and cuff can inflate the cuff and causes the cuff to seal the airway tube within a patient's trachea. The pilot cuff has a one way air valve, a syringe adapter, a balloon, and an inflating tube port. The syringe adapter is attached to the bracket. Forcing air into the syringe adapter opens the one way air valve and inflates the balloon. The inflating tube is attached to the inflating tube port such that air passing through the syringe adapter passes through the inflating tube. The inflating tube is also attached to the cuff that is located at the patient end of the airway tube. The air passing into the syringe adapter passes through the inflating tube and inflates the cuff to thereby seal the airway tube in an airway such as patient's trachea.

It is a yet further aspect that breaking a syringe tab can free an air syringe plunger. Pressing the syringe plunger into the air syringe can inflate the cuff if the air syringe is engaged to the syringe adapter.

### BRIEF DESCRIPTION OF THE FIGURES

The accompanying figures, in which like reference numerals refer to identical or functionally-similar elements throughout the separate views and which are incorporated in and form a part of the specification, further illustrate the present embodiments and, together with the detailed description of the embodiments, serve to explain the principles of the present embodiments. The figures are not necessarily to scale or full scale.

FIG. 1 illustrates an oral airway securing device in accordance with aspects of the embodiments;

FIG. 2 illustrates a second view of the oral airway securing device of FIG. 1 in accordance with aspects of the embodiments;

FIG. 3 illustrates a third view of the oral airway securing device of FIG. 1 in accordance with aspects of the embodiments;

FIG. 4 illustrates a stylet in accordance with aspects of the embodiments;

FIG. 5 illustrates a multifunction adapter in accordance with aspects of the embodiments;

FIG. 6 illustrates a second view of the multifunction adapter of FIG. 5 in accordance with aspects of the embodiments;

FIG. 7 illustrates elements for inflating and deflating the cuff in accordance with aspects of the embodiments;

FIG. 8 also illustrates elements for inflating and deflating the cuff in accordance with aspects of the embodiments;

FIG. 9 illustrates a multifunction adapter and pilot cuff in accordance with aspects of the embodiments;

FIG. 10 illustrates a multifunction adapter, syringe adapter, and one way air valve in accordance with aspects of the embodiments;

FIG. 11 illustrates a stylet in an airway tube in accordance with aspects of the embodiments;

FIG. 12 illustrates an airway tube in accordance with aspects of the embodiments; and

FIG. 13 illustrates another view of the airway tube of FIG. 12 in accordance with aspects of the embodiments.

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative embodiments of the embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout. As used herein, the term “and/or” includes any and all combinations of one or more of the associated listed items.

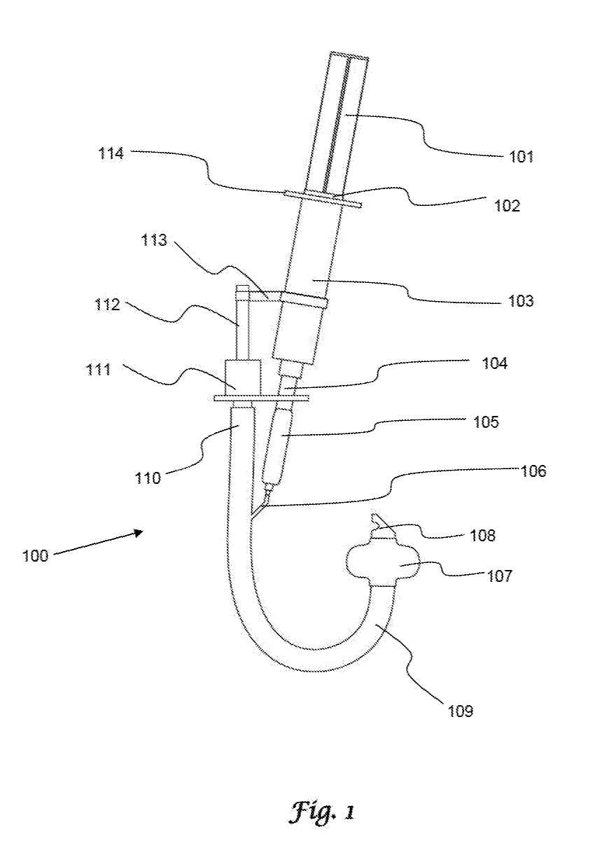

FIG. 1 illustrates an oral airway securing device 100 in accordance with aspects of the embodiments. An airway tube 109 has a patient end 108 and adapter end 110. The adapter end is attached to the proximal attachment of a multifunction adapter 111. The airway tube 109 is a flexible hose that is typically pressed over a cylindrical proximal attachment. A stylet 112 is installed in the airway tube 109 and extends out of the distal attachment of the multifunction adapter 111. The stylet 112 has a handle 113 holding an air syringe. The stylet 112 and air syringe can be removed by pulling the handle 113. The air syringe can be removed from the handle and reattached to the syringe adapter 104.

The stylet 112 of FIG. 1 shows the handle 113 engaging the air syringe toward the center of the body 103 (actually a little lower). Alternative embodiments can have the handle engaging the air syringe at the top collar 114 or directly under the top collar 114 such that the air syringe does not slide within the handle when the handle is pulled from the endotracheal tube.

A tab 102 prevents the plunger 101 from being pressed into the body 103 of the air syringe. The air syringe can be operated by removing tab 102 and pressing the plunger 101 into the body 103, thereby forcing air into the syringe adapter 104, balloon 105 and inflating tube 106 to inflate the cuff 107. The cuff 107 is shown inflated for illustration purposes because a deflated cuff would appear to be a widened area of the airway tube 109. Those practiced in the art of intubation know that a fully extended plunger 101, as illustrated, generally corresponds to a deflated cuff because, in practice, the cuff 107 is deflated by pulling the plunger 101 from the body 103.

The tab 102 is illustrated as a removable ring and can have a thinned section that can be torn to remove the tab 102. Alternatively, the tab 102 can be a partial, cut, or split ring that can be pulled from the syringe without damaging the tab 102. A tab 102 that must be damaged when removed can provide a positive indication that the airway syringe has been used. In many embodiments, the tab 102 can be put back in place to again hold the plunger 101 in an extended position.

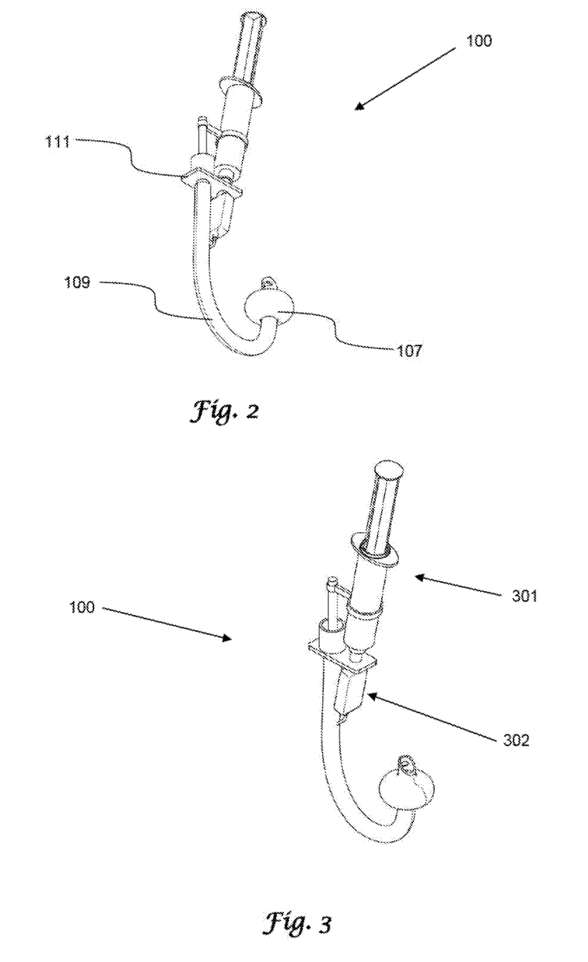

FIG. 2 illustrates a second view of the oral airway securing device 100 of FIG. 1 in accordance with aspects of the embodiments.

FIG. 3 illustrates a third view of the oral airway securing device 100 of FIG. 1 in accordance with aspects of the embodiments. This view more clearly shows aspects of the airway syringe 301 and pilot cuff 302. The pilot cuff 302 as illustrated is an assembly that includes the syringe adapter 104, balloon 105, inflating tube 106, and a one way air valve that isn't visible in FIG. 3.

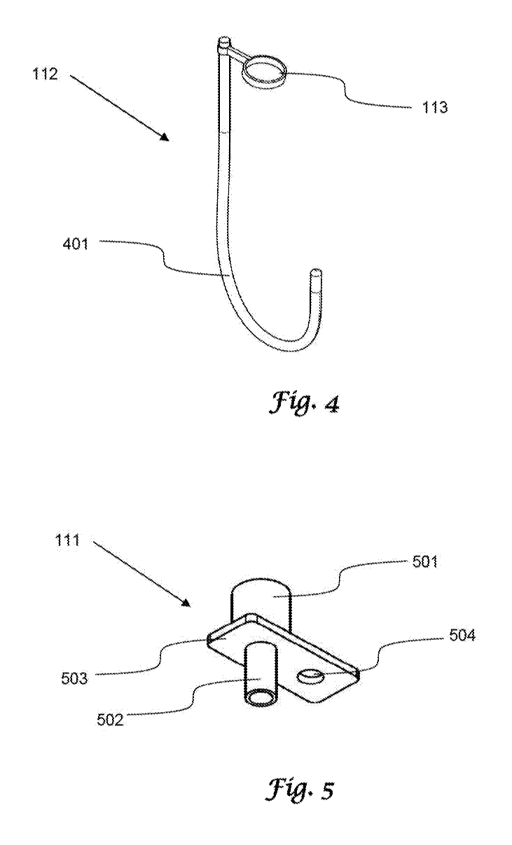

FIG. 4 illustrates a stylet 112 in accordance with aspects of the embodiments. The stylet 112 has a handle 113 and a posable length 401. The posable length is made from a material, such as a wire or metal tube that tends to remain in its bent shape after being bent. The airway tube 109 can therefore be bent into a desired shape when the posable length is inside the airway tube 109. As is notoriously well known the art, the stylet must be pulled from the airway tube 109 to fully secure a patient's airway.

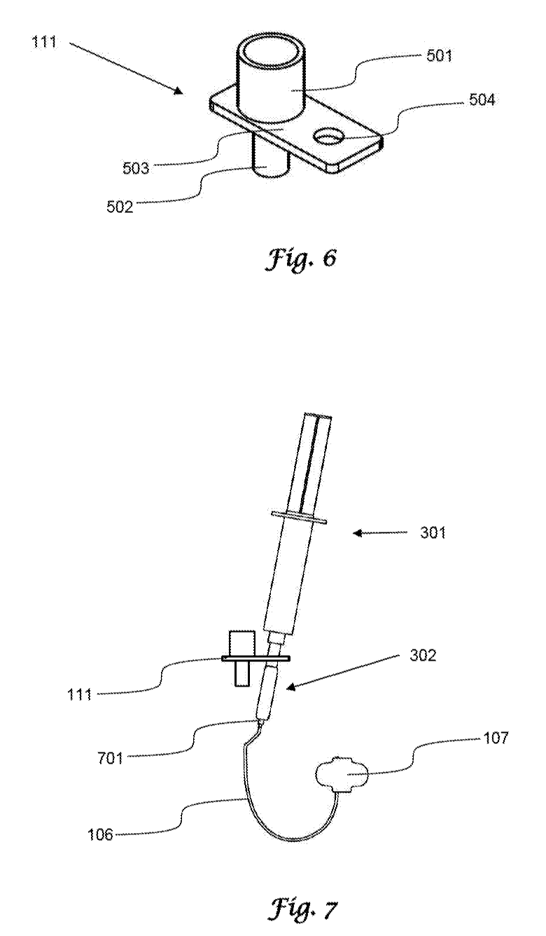

FIGS. 5 and 6 illustrate a multifunction adapter 111 in accordance with aspects of the embodiments. The multifunction adapter has a distal attachment 501 which is typically the commonly used 15 mm cylinder to which ventilation devices can be attached. The proximal attachment 502 is sized to fixedly engage the adapter end 110 of the airway tube 109. A bracket 503 between the proximal and distal attachments has an angled hole 504 that provides a mounting point for the pilot cuff 302. The syringe adapter 104 can be pressed into the angled hole 504 such that the pilot cuff 302 is held in place. Adhesive or glue can permanently attach the syringe adapter 104 to the multifunction adapter 111. Alternatively, the multifunction adapter 111 and syringe adapter can be produced as a single piece, perhaps as a single piece of molded plastic.

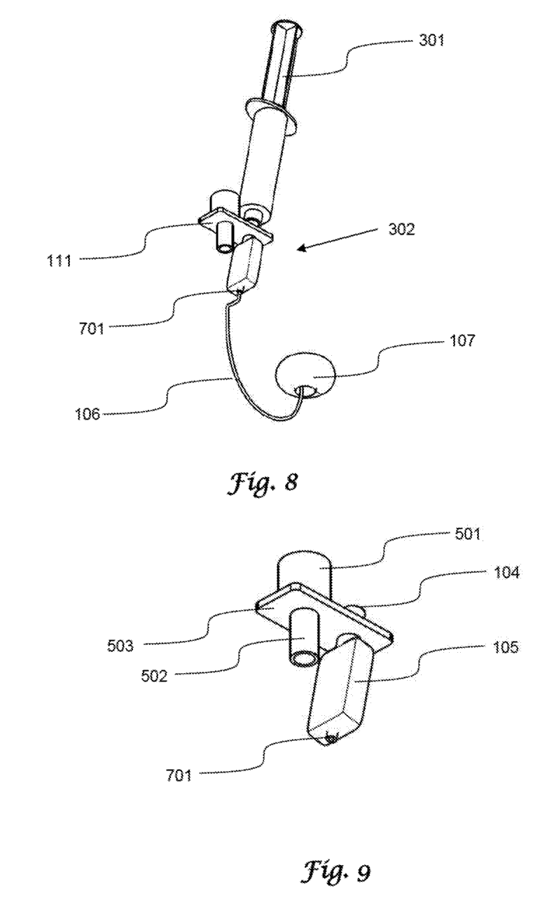

FIGS. 7 and 8 illustrate elements for inflating and deflating the cuff 107 in accordance with aspects of the embodiments. The air syringe 301 can force air into the pilot cuff 304, inflating tube 106, and cuff 107 to cause the cuff 107 to inflate and seal the airway tube in a patient's airway. As discussed above, the cuff 107 is shown inflated while the plunger 101 is in an extended position. In normal operation, the plunger 101 is pressed into the body 103 when the cuff 107 is inflated such that the plunger 101 can be pulled from the body 103 to deflate the cuff 107.

FIG. 9 illustrates a multifunction adapter and pilot cuff in accordance with aspects of the embodiments. This view provides a clearer showing of the inflating tube port 701. The inflating tube port 701 is the attachment point for the inflating tube 106.

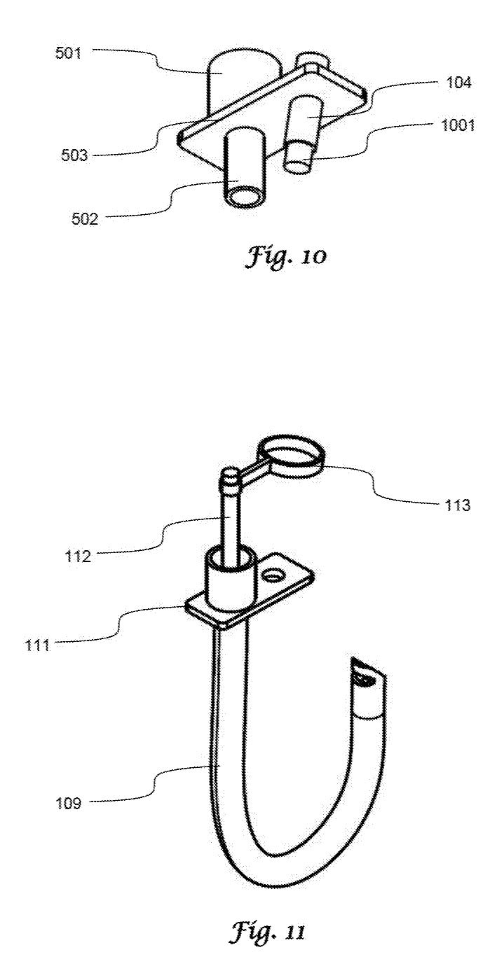

FIG. 10 illustrates a multifunction adapter, syringe adapter 104, and one way air valve 1001 in accordance with aspects of the embodiments. The one way air valve 1001 prevents air from passing from the balloon 105 and out the syringe adapter 104. It is the one way air valve 1001 that prevents the cuff 107 from deflating when the air syringe 301 is removed. The one way air valve can be pressed open by installing an air syringe 301 onto the syringe adapter 104 such that the air syringe 301 can be used to deflate the cuff 107.

FIG. 11 illustrates a stylet 112 in an airway tube 109 in accordance with aspects of the embodiments. The airway tube 109 can therefore be set into a configuration for intubating a patient. Here, configuration means the amount of curvature and where the curve is along the length of the airway tube 109. Medical care givers typically set the configuration based on a patients physiology.

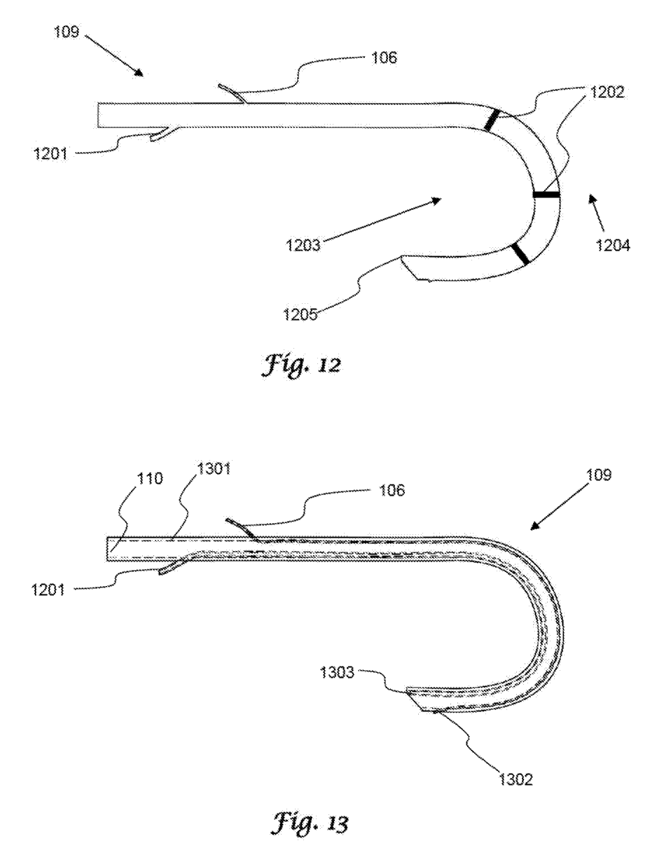

FIG. 12 illustrates an airway tube 109 in accordance with aspects of the embodiments. Unlike the previous illustrations, this embodiment has a suction tube 1201 and a differently placed inflating tube 106. Airway tubes typically have a curved shape with a concave side 1203 and a convex side 1204. Here, the visible end of the suction tube 1201 is on the convex side 1203 of the airway tube 109 and the air way tube 109 curves toward the visible end of suction tube 1201. Furthermore, the visible end of the inflating tube 106 is on the concave side 1204 of the airway tube 109 and the air way tube 109 curves away from the visible end of the inflating tube 106. Depth markers 1202 are also shown. Typically, numbers near the depth markers 12202 indicate the distance along the length of the airway tube 109 from the patient ends tip 1205.

FIG. 13 illustrates another view of the airway tube 109 of FIG. 12 in accordance with aspects of the embodiments. Here, the suction tube 1201 and the inflating tube 106 can be seen to pass through the sidewall 1301 of the airway tube 109 and run along the interior of the airway tube 109. Here, the inflating tube 106 exits back out of the sidewall 1301 at a cuff location 1302 that would be under a cuff, if installed. The suction tube 1201 proceeds to the end of the airway tube 109 with the end of the suction tube 1201 being a suction port 1303.

The previous discussion and associated figures have been directed to a particular endotracheal tube design. However, those practiced in the art realize that any other supraglottic airway such as the Laryngeal Mask Airway (LMA) and King LT airway could have been used in the illustrations. As such the discussion of the multifunction adapter and other aspects of the embodiments apply to those other supraglottic airways as well.

The foregoing description of the embodiments has been provided for purposes of illustration and description. It is not intended to be exhaustive or to limit the disclosure Individual elements, or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An oral airway securing device comprising:
a multifunction [15 mm] adapter comprising a distal attachment, a proximal attachment, and a bracket wherein the bracket is between the distal attachment, and the proximal attachment and wherein air passing into the distal attachment, passes though the bracket and into the proximal attachment;
a pilot cuff comprising a one way air valve, a syringe adapter, a balloon, and an inflating tube port wherein the syringe adapter is attached to the bracket, and wherein forcing air into the syringe adapter opens the one way air valve and inflates the balloon;
an inflating tube attached to the inflating tube port such that air passing through the syringe adapter passes through the inflating tube;
an airway tube comprising a patient end and an adapter end, wherein the adapter end is connected to the proximal attachment such that air passing through the distal attachment passes through the airway tube; and
a cuff located at the patient end of the airway tube and attached to the inflating tube, wherein the cuff encompasses the airway tube, wherein air passing into the syringe adapter passes through the inflating tube and inflates the cuff to thereby seal the airway tube in an airway such as a trachea.

2. The oral airway securing device of claim 2 wherein the bracket further comprises a distal face and further comprises an angled hole around an angled centerline, wherein the angled centerline is offset at least 5 degrees from a normal to the distal face, and wherein the syringe adapter is fixedly attached within the angled hole.

3. The oral airway securing device of claim 2 wherein the airway tube is closer to the inflating tube port than to the syringe adapter.

4. The oral airway securing device of claim 1 wherein the bracket further comprises the syringe adapter.

5. The oral airway securing device of claim 1 wherein the distal attachment a cylindrical tube having a centerline and wherein the bracket rotates at least 80 degreed around the centerline.

6. The oral airway securing device of claim 1 wherein the inflating tube passes through a sidewall of the airway tube within 50 mm of the bracket.

7. The oral airway securing device of claim 6 wherein the airway tube forms a curve when at rest and wherein the inflating tube passes through the sidewall of the airway tube along a concave portion of the curve.

8. The oral airway securing device of claim 6 wherein the airway tube forms a curve when at rest and wherein the inflating tube passes through the sidewall of the airway tube along a convex portion of the curve.

9. The oral airway securing device of claim 1 wherein the airway tube forms a curve when at rest and wherein the syringe adapter is attached to the bracket such that the airway tube curves toward the pilot cuff.

10. The oral airway securing device of claim 1 wherein the airway tube forms a curve when at rest and wherein the syringe adapter is attached to the bracket such that the airway tube curves toward the pilot cuff. An oral airway securing device, comprising:
a multifunction adapter comprising a distal attachment, a proximal attachment, and a bracket wherein the bracket is between the distal attachment, and the proximal attachment and wherein air passing into the distal attachment passes though the bracket and into the proximal attachment;
a pilot cuff comprising a one way air valve, a syringe adapter, a balloon, and an inflating tube port wherein the syringe adapter is attached to the bracket, and wherein forcing air into the syringe adapter opens the one way air valve and inflates the balloon;
an inflating tube attached to the inflating tube port such that air passing through the syringe adapter passes through the inflating tube;
an airway tube comprising a patient end and an adapter end, wherein the adapter end is connected to the proximal attachment such that air passing through the distal attachment passes through the airway tube; and
a cuff located at the patient end of the airway tube and attached to the inflating tube, wherein air passing into the syringe adapter passes through the inflating tube and inflates the cuff to thereby seal the airway tube in an airway such as a trachea.

12. The oral airway securing device of claim 11 further comprising a suction port wherein a suction tube passes into a sidewall of the airway tube at a sidewall entrance and lies within the airway tube from the sidewall entrance to the patient end.

13. The oral airway securing device of claim 11 further comprising a plurality of depth markers on the airway tube indicating a distance from the patient end.

14. The oral airway securing device of claim 11 further comprising a removable stylet wherein the stylet comprises a posable length and a handle, wherein the posable length passes though the distal attachment and into the airway tube such that a length of the airway tube is posable, and wherein the handle is configured to hold an air syringe in engagement with the syringe adapter.

15. The oral airway securing device of claim 14 wherein disengaging the stylet from the airway tube causes the syringe to disengage from the syringe adapter.

16. The oral airway securing device of claim 14 wherein the handle comprises a syringe holder that removably holds the air syringe.

17. The oral airway securing device of claim 11 further comprising an air syringe and a syringe tab wherein the air syringe comprises a body and a plunger and wherein the syringe tab holds the plunger a position relative to the body, wherein the, plunger is freed by removing the syringe tab, and wherein removing the syringe tab permanently alters the syringe tab.

18. The oral airway securing device of claim 11:
wherein the bracket further comprises a distal face and further comprises an angled hole around an angled centerline, wherein the angled centerline is offset at least 5 degrees from a normal to the distal face, wherein the syringe adapter is fixedly attached to within the angled hole, wherein airway tube is closer to the inflating tube port than to the syringe adapter, wherein the bracket further comprises the syringe adapter;
wherein the distal attachment is a cylindrical tube having a centerline and wherein the bracket rotates at least 80 degrees around the centerline;
wherein the inflating tube passes through a sidewall of the airway tube within 50 mm of the bracket; and
wherein the airway tube forms a curve when at rest and wherein the inflating tube passes through the sidewall of the airway tube along a convex portion of the curve and wherein the syringe adapter is attached to the bracket such that the airway tube curves toward the pilot cuff.

19. A method for securing a patients airway, the method comprising, passing a patient end of an airway tube into the patient's mouth and thence to the patient's trachea wherein the airway tube comprises a patient end and an adapter end, wherein the adapter end is attached to a proximal attachment of a multifunction adapter comprising a distal attachment, the proximal attachment, and a bracket wherein the bracket is between the distal attachment and the proximal attachment, and wherein air passing into the distal attachment passes though the bracket, through the proximal attachment, and into the airway tube;
forcing air into a pilot cuff, inflating tube, and cuff such that the cuff inflates to seal the airway tube within a trachea;
wherein the pilot cuff comprises a one way air valve, a syringe adapter, a balloon, and an inflating tube port, wherein the syringe adapter is attached to the bracket, and wherein forcing air into the syringe adapter opens the one way air valve and inflates the balloon;
wherein the inflating tube is attached to the inflating tube port such that air passing through the syringe adapter passes through the inflating tube; and
wherein the inflating tube is also attached to the cuff, wherein the cuff is located at the patient end of the airway tube, wherein air passing into the syringe adapter passes through the inflating tube and inflates the cuff to thereby seal the airway tube in an airway such as a trachea.

20. The method for securing a patients airway of claim 19 further comprising:
breaking a syringe tab to thereby free a syringe plunger; and
pressing the syringe plunger into an air syringe wherein the air syringe is engaged to the syringe adapter to thereby inflate the cuff.
